(19) United States
(12) Patent Application Publication
(10) Pub. No.: US 2011/0264113 A1
(43) Pub. Date: Oct. 27, 2011

(54) SURGICAL INSTRUMENT

(76) Inventors: Seung Wook CHOI, Seongnam-si (KR); Jong Seok Won, Yongin-si (KR); Jae Sun Lee, Seongnam-si (KR)

(21) Appl. No.: 13/176,414

(22) Filed: Jul. 5, 2011

Related U.S. Application Data
(62) Division of application No. 13/129,334, filed on May 13, 2011.

(30) Foreign Application Priority Data
Dec. 12, 2008 (KR) 10-2008-0126415
Dec. 30, 2008 (KR) 10-2008-0136840
Dec. 30, 2008 (KR) 10-2008-0136859
Jan. 21, 2009 (KR) 10-2009-0004872

(51) Int. Cl. A61B 19/00

(52) U.S. Cl. 606/130

(57) ABSTRACT

A surgical instrument is disclosed. The surgical instrument, which has an effector for engaging the surgical site joined to one end and a driving part for operating the effector joined to the other end, includes: a first shaft, which has one end joined with the driving part, and which extends along a first lengthwise direction; and a second shaft, which extends along a second lengthwise direction that forms a particular angle with the first shaft, and which has one end joined with the other end of the first shaft such that the second shaft is rotatable about an axis following the second lengthwise direction. Thus, it is possible to conduct surgery using several of such surgical instruments without having the instruments obstruct one another, and the surgical instrument can be made to have different usage modes according to what length it is set to.

### CROSS-REFERENCE TO RELATED APPLICATIONS

This application is a Divisional Application of co-pending U.S. patent application No. 13/129,334 filed May 13, 2011, which is the National Phase of PCT/KR2009/007290 filed on Dec. 8, 2009, which claims priority under 35 U.S.C. 119(a) to Patent Application No. 10-2008-0126415 filed in the Republic of Korea on Dec. 12, 2008, Patent Application No. 10-2008-0136859 filed in the Republic of Korea on Dec. 30, 2008, Patent Application No. 10-2008-0136840 filed in the Republic of Korea on Dec. 30, 2008, and Patent Application No. 10-2009-0004872 filed in the Republic of Korea on Jan. 21, 2009. All of those applications are hereby expressly incorporated by reference into the present application.

### BACKGROUND

The present invention relates to a medical apparatus, more particularly to a surgical instrument.

In the field of medicine, surgery refers to a procedure in which a medical apparatus is used to make a cut or an incision in or otherwise manipulate a patient's skin, mucosa, or other tissue, to treat a pathological condition. A surgical procedure such as a laparotomy, etc., in which the skin is cut open and an internal organ, etc., is treated, reconstructed, or excised, may entail problems of blood loss, side effects, pain, and scars. Thus, current methods of surgery that involve making an incision in the skin and inserting only a medical apparatus, such as a laparoscope, a surgical instrument, and a microscope, for example, or those that involve the use of surgical robots are currently regarded as popular alternatives.

A set of surgical robots may include a master robot, which is manipulated by the doctor to generate and transmit the necessary signals, and a slave robot, which receives the signals from the master robot to actually apply the manipulation to the patient. The master robot and the slave robot can be arranged in the operating room as an integrated unit or as separate devices.

A slave robot may be equipped with a robot arm to make manipulations for surgery, while an instrument may be mounted on the front end of the robot arm. As illustrated in FIG. 1, a conventional instrument 54 for mounting on a robot arm may include a driving part 108, a shaft 102 extending from the driving part 108, and a forceps-like effector 112 mounted on the far end 106 of the shaft 102 that is to be inserted into the surgical site.

A conventional instrument 54 may have an adapter part that joins to the robot arm. The driving forces may be transferred from the robot arm to rotate the driving wheels (not shown) that are pulley-joined by wires to the respective parts of the effector 112, and as a result, the parts of the effector 112 may be moved. In the case of a manually operated instrument, the surgeon may manipulate the driving part to move the effector, when holding or cutting the surgical site.

However, for a type of surgery that is performed with just one incision made in the surgical site, such as single port access (SPA) surgery and microsurgery, etc., a conventional surgical instrument may not be used with a high level of freedom, since a laparoscope and the instrument may all be inserted through the one incision for surgery.

The information in the background art described above was obtained by the inventors for the purpose of developing the present invention or was obtained during the process of developing the present invention. As such, it is to be appreciated that this information did not necessarily belong to the public domain before the patent filing date of the present invention.

### SUMMARY

An aspect of the present invention is to provide a surgical instrument that can be used in multiple numbers simultaneously without interfering with or obstructing one another and can be manipulated intuitively as if the surgeon were using one's own hands.

Other technical problems addressed by the present invention will be readily understood from the descriptions that follow.

One aspect of the present invention provides a surgical instrument that includes: a driving part; a shaft joined to the driving part that extends along one direction and has an elbow formed in the middle; and an effector joined to the far end of the shaft that operates in correspondence with a user manipulation on the driving part, where the shaft can be configured to curve at the elbow.

The elbow can include a hinge axis, formed on one side as seen from a cross section of the shaft, and an expandable part, formed on the other side of the cross section of the shaft, where the shaft can be configured to curve at the hinge axis in a direction that compresses the expandable part. The expandable part can include an elastic body that applies an elastic force in a direction that expands the expandable to straighten the shaft or compresses the expandable part to curve the shaft. In this case, the driving part can include a driver, with a wire connecting the driver with a particular point in a vicinity of the elbow, and the shaft can be curved at the elbow by manipulating the driver to apply a tensional force on the wire.

There can be a multiple number of elbows formed in the shaft, and the elbows can be formed to curve the shaft in opposite directions, so that the effector may move closer to the driving part as the shaft is curved.

The shaft can include a core and a guide member, where the core may be made from a flexible material, and the guide member may surround the core, with the elbow formed in a portion of the guide member. Thus, the core can be curved as the guide member is curved. In this case, the guide member can be used as a surgical trocar.

A wire can be connected to a point near the guide member, and by applying a tensional force on the wire, the guide member may be curved at the elbow. A driving wheel may be joined to the guide member, and the wire may be connected to the driving wheel, where a tensional force can be applied on the wire by manipulating the driving wheel. In this case, the driving part can include a driver, with the driving wheel connected to the driver, to be manipulated in linkage with a manipulation of the driver.

The wire can be installed exposed at a surface of the shaft, and the wire may be pulled out of the shaft as a tensional force is applied on the wire to curve the shaft. In this case, the shaft can have a cylindrical shape, and the wire can form a portion of the perimeter of the shaft. Also, the shaft can be formed with a channel processed in its cross section to hold the wire.

The driving part can be coupled to a surgical robot arm to be manipulated by a driving force transferred from the robot, or alternatively, can be formed as a handle to be manually manipulated by a user.

Another aspect of the present invention provides a master interface for a surgical robot. The master interface is mounted on a master robot and enables a user to conduct robotic surgery by manipulating a surgical instrument mounted on a slave robot connected to the master robot. This master interface includes an elbow handle that generates a particular manipulation signal for operating the instrument, where an elbow is formed in a shaft of the instrument, the shaft is configured to curve at the elbow, and the elbow handle is configured to generate the manipulation signal for curving the shaft. In this case, the elbow handle can be worn on an elbow of a user, to be operated in accordance with the movement of the user's elbow.

Yet another aspect of the present invention provides a method of driving a surgical instrument mounted on a slave robot by connecting the slave robot to a master robot and manipulating the master robot. This method includes: generating a particular manipulation signal in correspondence with the movement of an elbow handle, which is included on the master robot, and which is worn on an elbow of a user; converting the manipulation signal into a driving signal that corresponds to a curving operation of a shaft of the instrument; and transmitting the driving signal to the slave robot. After the transmitting, the method can further include: curving the shaft to correspond with a movement of the elbow of the user, using the driving signal.

The general and specific aspects above can be implemented as a system, method, or a computer program, or as any combination of systems, methods, and computer programs.

Additional aspects, features, and advantages, other than those described above, will be obvious from the claims and written description below.

Certain embodiments of the present invention make it possible to conduct surgery using several surgical instruments without having the instruments obstruct one another, and a surgical instrument can be made to have different usage modes according to what length it is set to.

Also, by forming an elbow in the shaft of a surgical instrument and enabling the shaft to bend according to a manipulation on the driving part, the shaft of the instrument can be made to perform articular movements similar to those of a wrist or an elbow. Thus, a surgeon may manipulate the instrument intuitively, just as if the surgeon were using his or her own hands.

Since the shaft of the instrument can be bent as necessary, several instruments can be inserted from different directions through a single insertion hole, and for each instrument, the shaft can be bent such that the effector faces a particular surgical site. Thus, even when using more than one instruments at once, the instruments may not interfere with or obstruct one another, and an effective mode of “minimally invasive surgery” can be implemented.

### BRIEF DESCRIPTION OF THE DRAWINGS

FIG. 1 is a perspective view of a surgical instrument according to the related art.

FIG. 2 is a diagram schematically illustrating a surgical instrument according to an embodiment of the present invention.

FIG. 3 is a magnified view of the elbow portion of a surgical instrument according to an embodiment of the present invention.

FIG. 4 is a diagram illustrating the operation of a surgical instrument according to an embodiment of the present invention.

FIG. 5 is a diagram illustrating the operation of a surgical instrument according to another embodiment of the present invention.

FIG. 6 is a diagram illustrating the operation of a surgical instrument according to another embodiment of the present invention.

FIG. 7 is a diagram illustrating possible cross sections for the shaft of a surgical instrument according to an embodiment of the present invention.

FIG. 8 is a diagram illustrating the composition of a surgical robot according to an embodiment of the present invention.

FIG. 9 is a perspective view of a master interface for a surgical robot according to an embodiment of the present invention.

FIG. 10 is a flowchart illustrating a method of driving a surgical robot according to an embodiment of the present invention.

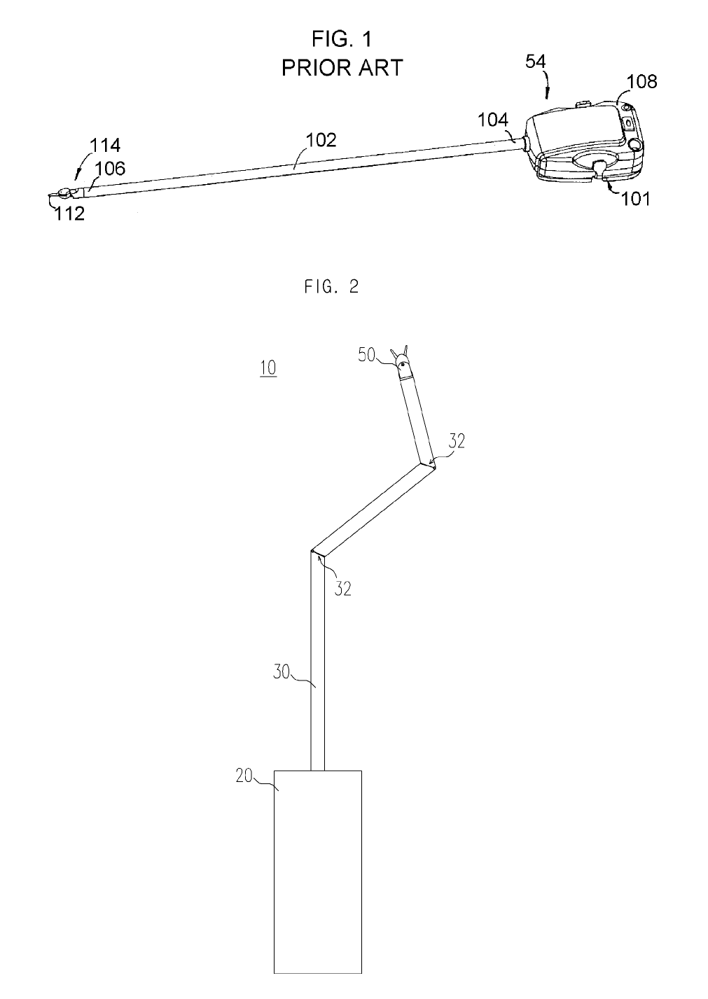

### DETAILED DESCRIPTION

As the present invention allows for various changes and numerous embodiments, particular embodiments will be illustrated in the drawings and described in detail in the written description. However, this is not intended to limit the present invention to particular modes of practice, and it is to be appreciated that all changes, equivalents, and substitutes that do not depart from the spirit and technical scope of the present invention are encompassed in the present invention.

While terms including ordinal numbers, such as “first” and “second,” etc., may be used to describe various components, such components are not limited to the above terms. The above terms are used only to distinguish one component from another. For example, a first component can be referred to as a second component without departing from the scope of claims of the present invention, and likewise, a second component can be referred to as a first component. If a component is said to be “connected to” or “accessing” another component, it is to be appreciated that the two components can be directly connected to or directly accessing each other but can also include one or more other components in-between.

The terms used in the present specification are merely used to describe particular embodiments, and are not intended to limit the present invention. An expression used in the singular encompasses the expression of the plural, unless it has a clearly different meaning in the context. In the present specification, it is to be understood that the terms “including” or “having,” etc., are intended to indicate the existence of the features, numbers, steps, actions, components, parts, or combinations thereof disclosed in the specification, and are not intended to preclude the possibility that one or more other features, numbers, steps, actions, components, parts, or combinations thereof may exist or may be added.

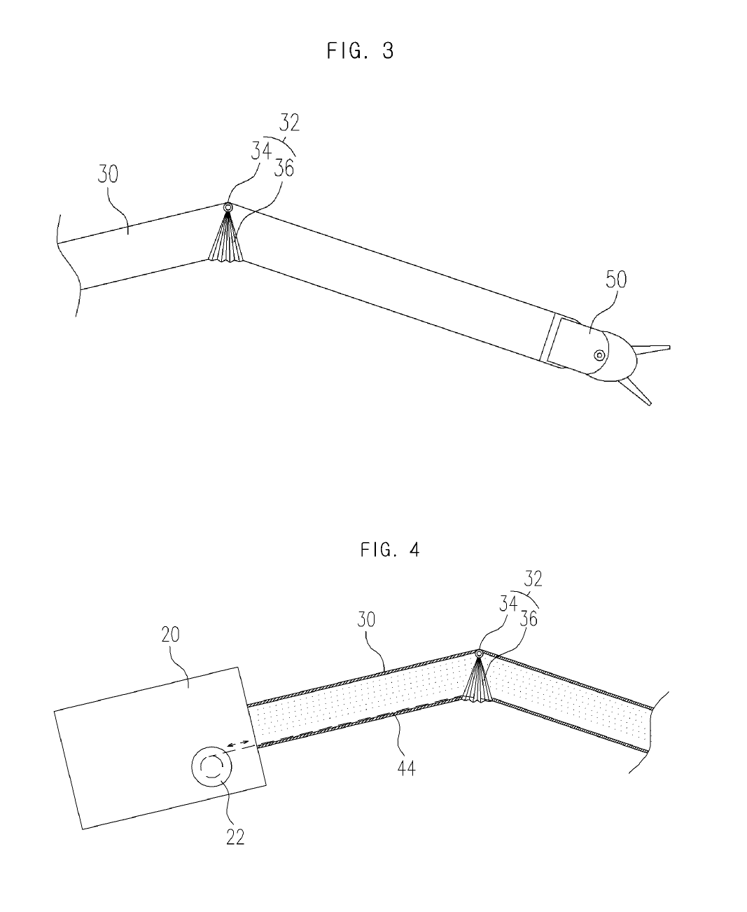

FIG. 2 is a diagram schematically illustrating a surgical instrument according to an embodiment of the present invention, and FIG. 3 is a magnified view of the elbow portion of a surgical instrument according to an embodiment of the present invention. Illustrated in FIG. 2 and FIG. 3 are an instrument 10, a driving part 20, a shaft 30, elbows 32, a hinge axis 34, an expandable part 36, and an effector 50.

A feature of this embodiment is that an elbow structure is applied to the middle of the shaft 30 in the surgical instrument, so that the shaft 30 may be curved in the middle. Thus, when the far end of the shaft 30, i.e. the effector 50, is inserted into the body during a surgical procedure, a surgeon may manipulate the surgical instrument just as if the surgeon's own arms are moved inside the body.

An instrument 10 according to this embodiment can be composed mainly of a driving part 20, a shaft 30 extending in one direction from the driving part 20, and an effector 50 joined to the far end of the shaft 30. In the case of a robotic surgical instrument, the driving part 20 may be the part that is mounted on a surgical robot to receive driving forces transferred from the surgical robot, and in the case of a manually operated instrument, the driving part 20 may be the part that is held and manipulated by the user to receive its driving forces directly from the hands of the user.

Onto this driving part 20, a driving wheel or driver can be installed which engages an actuator of the robot, or a handgrip such as a wheel, lever, switch, etc., can be installed which may be held by the user. When a driving force is transferred from the robot, or when the user manually manipulates the driving part 20, the effector 50 may accordingly move in a gripping, rotating, tilting movement, etc., to implement a maneuver required for surgery.

In other words, the driving part 20 according to this embodiment can be configured to couple onto a surgical robot arm and be manipulated by driving forces transferred from the robot, in the case of a robotic surgical instrument, and can be configured to be manually manipulated by the user, in the case of a manually operated instrument.

The shaft 30 can be shaped as a straight line extending in one direction, and by using a tube member having a typical cylindrical shape, etc., the shaft 30 can hold the pulley-wires that connect the driving part 20 with various portions of the effector 50 to transfer the driving forces from the driving part 20 to the effector 50. Thus, when portions of the driving part 20 are manipulated, the respective portions of the effector 50 connected by pulley-wires may be moved.

As illustrated in FIG. 2, the shaft 30 of an instrument 10 according to this embodiment can have elbows 32 formed in the middle, enabling the shaft 30 to curve at the elbows 32. An elbow 32 may serve as an articulation at which the straight shaft 30 may bend by a particular angle. The function of the elbow 32 can be implemented by forming the elbow 32 portion, or the entire shaft 30, in the shape of a corrugated tube or bellows.

As illustrated in FIG. 3, an elbow 32 according to this embodiment can be composed with a hinge axis 34 formed on one side and an expandable structure on the other, when looking at the cross section of the shaft 30. In this way, the shaft 30 may be curved at the elbow 32, to be more specific, at the hinge axis 34, in a direction that contracts the expandable part 36. Thus, for a shaft 30 according to this embodiment, the direction and the degree in which the shaft 30 is curved can be determined by the structure of the elbows 32 formed in the middle.

The expandable part 36 is a component that enables to shaft 30 to bend or unbend while maintaining its shape. The expandable part 36 can be shaped as a corrugated tube or bellows, or can be made from a flexible material.

Furthermore, the expandable part 36 can include an elastic body that applies an elastic force in a direction that expands the expandable part. That is, an elastic body such as a spring, etc., can be included in the expandable part, while a stopper, etc., can be formed in the hinge axis to prevent the expandable part from expanding excessively. Then, the shaft may normally remain in a straight, unbent state, but when it is pulled using a wire, etc., the expandable part may contract and the shaft may bend at the elbow, and when the tensional force on the wire is removed, the shaft may return to its unbent state due to the restoring force of the elastic body.

Alternatively, the expandable part 36 can include an elastic body such as a spring, etc., that applies an elastic force in a direction that contracts the expandable part. Then, the shaft may normally (when there is no force applied) remain in a bent state, but when a force is applied using a wire, etc., the expandable part may expand and the shaft may be unbent into a straight form, and when the external force is removed, the shaft may return to its bent state due to the restoring force of the elastic body. Such configurations can be used to improve safety during surgical procedures.

A description will now be provided as follows on the operation of an instrument 10 according to this embodiment, using an example that includes the elbow structure illustrated in FIG. 3.

FIG. 4 is a diagram illustrating the operation of a surgical instrument according to an embodiment of the present invention. Illustrated in FIG. 4 are a driving part 20, a driver 22, a shaft 30, an elbow 32, a hinge axis 34, an expandable part 36, and a wire 44.

A shaft 30 in which an elbow 32 is formed according to this embodiment can be operated by the tension of the wire 44. That is, a wire 44 can be connected near the elbow 32 and connected to the driving part 20, whereby the shaft 30 can be made to fold at the elbow 32 by manipulating the driving part 20 to apply a tensional force on the wire 44.

Referring to the portion of the driving part 20 where the wire 44 is connected as the driver 22, the shaft 30 of an instrument 10 according to this embodiment may be curved at the elbow 32 according to the manipulation of the driver 22. The driving part 20 can be equipped with other drivers 22 for operating the effector 50, and these other drivers 22 can be connected with other wires, which connect to the effector 50. Details on the structure, function, operating method, etc., of the drivers 22 and wires for operating the effector 50 will be omitted here, and in the descriptions that follow, the terms “driver” and “wire” will refer to the driver 22 and wire 44 for curving the shaft 30, respectively, unless otherwise stated.

As already described above, a shaft 30 according to this embodiment can be made from a tube-shaped member having a typical cylindrical shape, etc. In this case, the wire 44 may be held within the shaft 30 and extend along the lengthwise direction of the shaft 30 to be connected to a particular position near the elbow 32.

As illustrated in FIG. 4, a shaft 30 according to this embodiment can include a multiple number of elbows 32. For example, if a shaft 30 according to this embodiment were to be compared to a human arm, the elbows 32 illustrated in FIG. 4 can be regarded as corresponding to the elbow and wrist joints.

In certain cases where the effector 50 joined to the end of the shaft 30 is to be drawn close to or away from the driving part 20 by curving the shaft 30, it is possible to form the structure of the elbows 32 such that the shaft 30 is folded in a zigzag shape, i.e. with each elbow curving the shaft in opposite directions. Thus, just as a person is able to move one's hand closer to or further from the shoulder according to the movement of the elbow and wrist joints, the effector 50 can be moved closer to or further from the driving part 20 by bending or unbending the shaft 30 at each of the elbows 32.

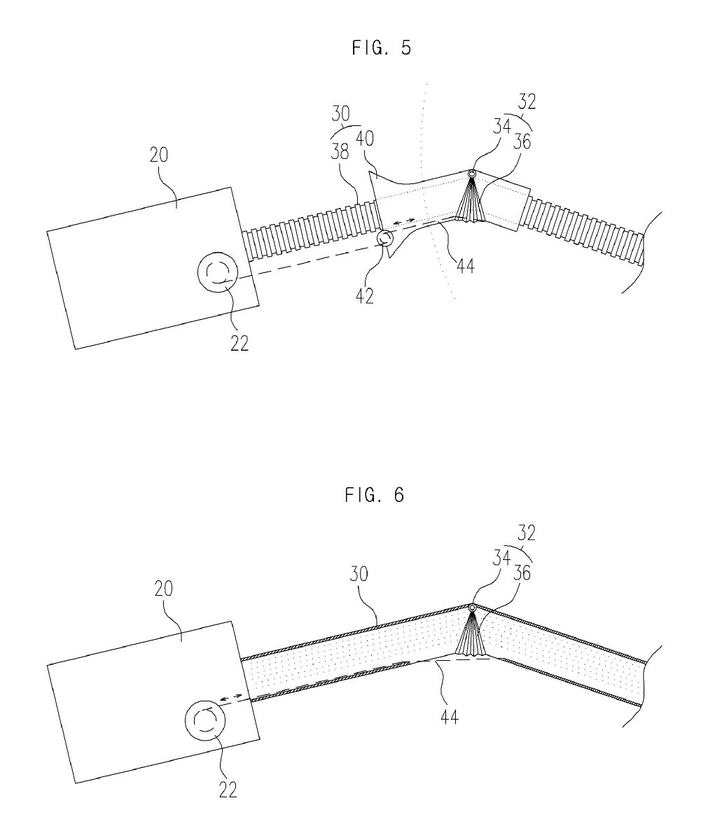

FIG. 5 is a diagram illustrating the operation of a surgical instrument according to another embodiment of the present invention. Illustrated in FIG. 5 are a driving part 20, a driver 22, a shaft 30, an elbow 32, a hinge axis 34, an expandable part 36, a core 38, a guide member 40, a driving wheel 42, and a wire 44.

This embodiment relates to forming the shaft 30 as a dual structure, i.e. including an inner core 38 that serves as a channel for holding the wire 44 and a guide member 40 that surrounds the core 38. The core 38 can be made from a flexible material, to be capable of bending freely, and the rigid guide member 40 can surround the perimeter of the core 38, with an elbow 32 such as that described above formed in the middle of the guide member 40. Thus, the core 38 can be curved, i.e. the shaft 30 can be curved, by curving the guide member 40.

In this case, the core 38 may be made from a material and/or structure, such as of a corrugated tube, etc., which is flexible but does not change shape unless an external force is applied. The core 38 may then maintain a certain shape (e.g. a straight line), until the guide member 40 is curved at the elbow 32, when the core 38 may change to a curved shape, after which the core 38 may remain in this changed state.

A guide member 40 according to this embodiment can also be used as a surgical trocar. In this case, the guide member 40 (trocar) may first be inserted into the surgical site, and then the core 38 of the instrument 10 may be inserted through the trocar, so that the core 38 inserted through the guide member 40 (trocar) may, as a whole, serve as the shaft 30. If the shaft 30 is to be curved to a particular angle, the guide member 40 may be bent at the elbow 32 formed in the guide member 40, causing the core 38 to change shape accordingly, and consequently causing the shaft 30 to curve.

For curving the guide member 40, it is possible to connect a wire 44 to the vicinity of the elbow 32 of the guide member 40 and apply a tensional force on the wire 44 to curve the guide member 40 at the elbow 32, similar to the previously described embodiments. Moreover, the guide member 40 can be made to curve at the elbow 32 due to the tension on the wire 44, by including a driver 22 in the driving part 20, connecting the wire 44 to the driver 22, and manipulating the driver 22.

It is also possible to join a separate driving wheel 42 to the guide member 40 and connect the wire 44 to the driving wheel 42, so that the guide member 40 may be curved when a tensional force is applied on the wire 44 according to the manipulation of the driving wheel 42. In cases where the guide member 40 is used as a trocar as described above, the instrument 10 may be inserted through the guide member 40, and afterwards the trocar, i.e. the guide member 40, can be bent by a particular angle by manipulating the driving wheel 42 joined to the guide member 40.

The manipulation for bending the guide member 40 after joining a separate driving wheel 42 can be performed manually, or the driving wheel 42 can be connected to the driver 22 included in the driving part 20, so that the driving wheel 42 may be manipulated in linkage with a manipulation on the driver 22. Of course, various mechanical connection methods, such as pulley-wires and links, etc., can be applied for linking the operation of the driving wheel 42 to that of the driver 22.

In such cases where a driving wheel 42 is joined to the guide member 40 and a driver 22 is included in the driving part 20, the driving wheel 42 can be made to operate in linkage with the manipulation of the driver 22 by connecting the driving wheel 42 with the driver 22 during or after the process of inserting the core 38 of the instrument 10 through the guide member 40.

FIG. 6 is a diagram illustrating the operation of a surgical instrument according to another embodiment of the present invention. Illustrated in FIG. 6 are a driving part 20, a driver 22, a shaft 30, an elbow 32, a hinge axis 34, an expandable part 36, and a wire 44.

The wire 44 used for applying a tensional force to curve the shaft 30 at the elbow 32 can be held within the shaft 30 as described above, but can also be exposed at the surface of the shaft 30, or configured to be pulled out of the shaft 30.

That is, if the wire 44 connecting the driver 22 with the elbow 32 is held inside the shaft 30, the process of curving the shaft 30 by applying tension on the wire 44 can entail an amount of friction generated between the wire 44 and the bent portion within the shaft 30. This may create a risk of damage to the wire 44 and/or the shaft 30 as well as a risk of malfunctioning in the curving operation.

To prevent such risks, a different material can be used for a portion of the shaft 30, or a separate bearing member, etc., can be used, to minimize friction between the wire 44 and the bent portion of the shaft 30. Alternatively, a portion of the can be uncovered, as illustrated in FIG. 6, so that the wire 44 may be pulled out of the shaft 30 when a tensional force is applied on the wire 44.

For example, a slit can be perforated in a portion of the shaft 30, and the shaft 30 can be installed in such a way that the wire 44 can be exposed through the slit at the surface of the shaft 30. Then, as the shaft 30 is curved, the wire 44 can be pulled out of the shaft 30 in correspondence to the shortest distance between the elbow 32 and the driving part 20, so that unnecessary friction between the wire 44 and the shaft 30 can be minimized, and the tensional force can be effectively delivered through the wire 44.

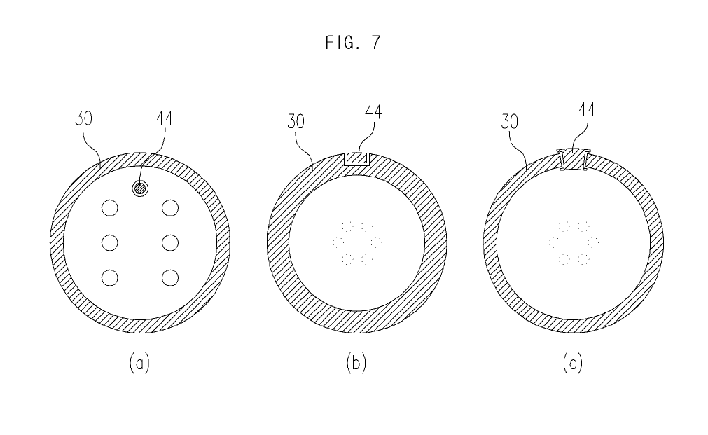

FIG. 7 is a diagram illustrating possible cross sections for the shaft of a surgical instrument according to an embodiment of the present invention. FIG. 7 shows illustrations of shafts 30 and wires 44.

The following relates to examples of cross sections for the shaft 30, in cases where the wire 44 is held inside the shaft 30 or exposed at the surface of the shaft 30, as mentioned with regard to the previously described embodiment.

Drawing (a) of FIG. 7 illustrates a shaft 30 having a circular cross section, where the channels for holding a multiple number of wires are perforated separately. Not only the wire 44 according to this embodiment but also other wires for operating the effector 50 can be held within the perforated channels. This allows the wires to effectively transfer the tensional forces generated according to the manipulation of the driving part 20 without interfering or causing friction with one another within the shaft 30.

Drawing (b) of FIG. 7 illustrates a shaft 30 having a circular cross section, where the wires for operating the effector 50 are held inside, and the wire 44 according to this embodiment is exposed at the surface of the shaft 30. In order to provide a smooth surface for the shaft 30, without having the wire 44 protrude out from the surface of the shaft 30, a portion of the exterior of the shaft 30 can be recessed to form a trough, such as that illustrated in drawing (b) of FIG. 7, and the wire 44 can be installed with a cross section corresponding with that of the trough.

Drawing (c) of FIG. 7 illustrates the cross section of a shaft 30 that is formed as a partially opened cylinder, where the wires for operating the effector 50 are held inside, and the wire 44 according to this embodiment is installed to cover the open portion of the shaft 30. That is, the wire 44 may form a portion of the perimeter of the shaft 30, so that normally, the wire 44 may close off the space within the shaft 30.

For the examples shown in drawings (b) and (c) of FIG. 7, the wire 44 may be pulled out of the shaft 30 when a tensional force is applied on the wire 44 to curve the shaft 30, as described above with reference to FIG. 6, so that unnecessary friction between the wire 44 and the shaft 30 can be minimized, and the tensional force can be effectively delivered through the wire 44.

Although it is not illustrated in the drawings, it is also conceivable, instead of using the tube-shaped shaft 30, to have the wire 44 according to this embodiment and the wires for operating the effector 50 combine together and form a cross section for a shaft 30. In this case, the wire 44 according to this embodiment can be exposed at the surface of the shaft 30 and may be naturally pulled out of the shaft 30 as the shaft 30 is curved.

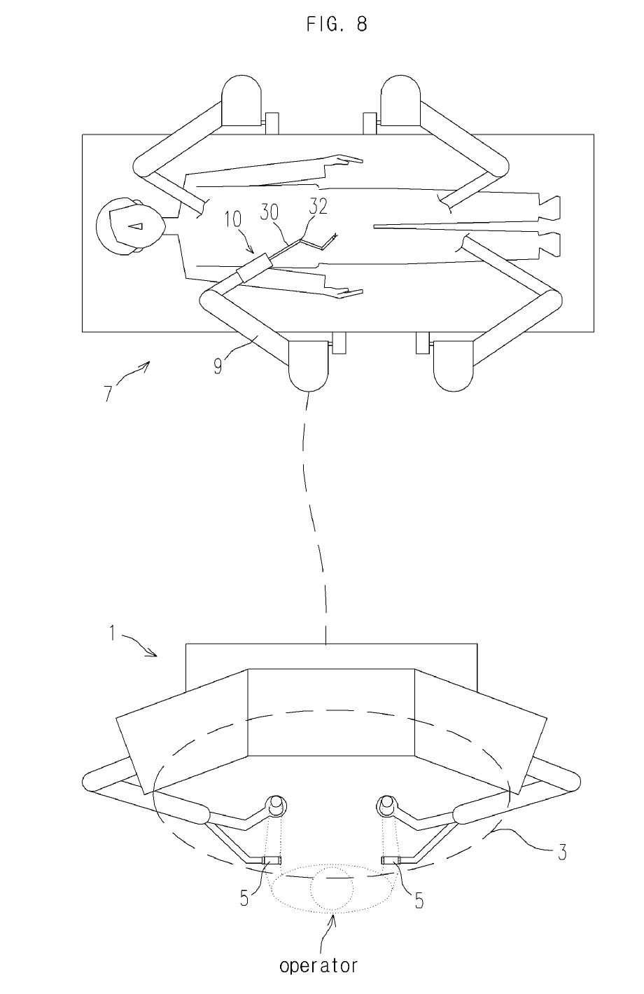

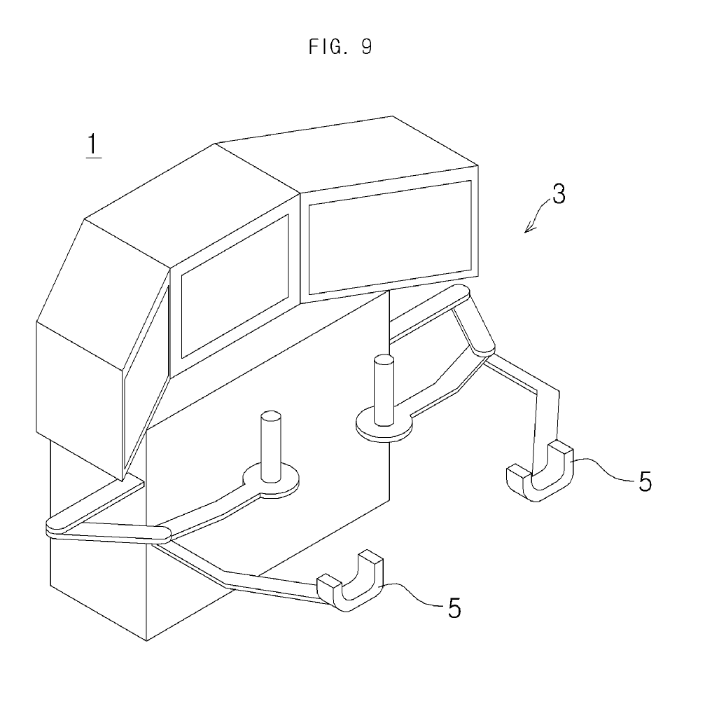

FIG. 8 is a diagram illustrating the composition of a surgical robot according to an embodiment of the present invention, and FIG. 9 is a perspective view of a master interface for a surgical robot according to an embodiment of the present invention. Illustrated in FIG. 8 and FIG. 9 are a master robot 1, an interface 3, elbow handles 5, a slave robot 7, robot arms 9, an instrument 10, a shaft 30, and an elbow 32.

This embodiment relates to a surgical robot that may be driven after mounting an instrument 10 described above, as well as to a master interface for the surgical robot. That is, as a means to make manipulations for curving the shaft 30 of the instrument 10, the master interface 3 may be equipped with handles dedicated to inputting these manipulations. A particular signal generated in accordance with a manipulation on the dedicated handles may be transferred to the slave robot 7 to correspond with a curving action of the shaft 30. In the descriptions that follow, these handles dedicated to this purpose will be referred to as “elbow handles.”

A surgical robot according to this embodiment may include a master robot 1 and a slave robot 7. An interface 3 that enables a user to make manipulations may be installed in the master robot 1, and when a manipulation is inputted, by way of various handles, levers, buttons, clutches, etc., equipped on the interface 3, a corresponding signal may be transmitted to the slave robot 7 and the slave robot 7 may be operated.

The slave robot 7 can be equipped with one or more robot arms 9, to which a surgical instrument 10 may be mounted. Each robot arm 9, as well as the instrument 10 mounted on the robot arm 9, may be driven according to a signal transmitted from the master robot 1 to conduct surgery.

On a master interface 3 according to this embodiment, a separate elbow handle 5 can be installed for generating a particular manipulation signal. As already described above, an instrument 10 according to this embodiment can include an elbow 32 formed in the shaft 30, and the shaft 30 can curve at the elbow 32, so the manipulation signal generated according to the manipulation of the elbow handle 5 may be transmitted to the slave robot 7 and used in curving the shaft 30 of the instrument 10.

As described above for the previously disclosed embodiments, a feature of an instrument 10 according to this embodiment is that the shaft 30 can be curved, in a manner analogous to an elbow joint. As such, the elbow handle 5 can be installed in a shape and structure that allows the elbow handle 5 to be worn on the elbow of the user. Then, the user may wear the elbow handle 5 on the elbow and move the elbow handle 5, causing the shaft 30 to operate in correspondence with the movement of the user's elbow.

For this purpose, an elbow handle 5 according to this embodiment can be formed as a U-shaped armrest into which the elbow portion of the user may be inserted. After inserting the elbow portion into this elbow handle 5, the user may manipulate the shaft 30 of the instrument 10 just as if the user were moving one's own arm, and the user may manipulate the robot more intuitively.

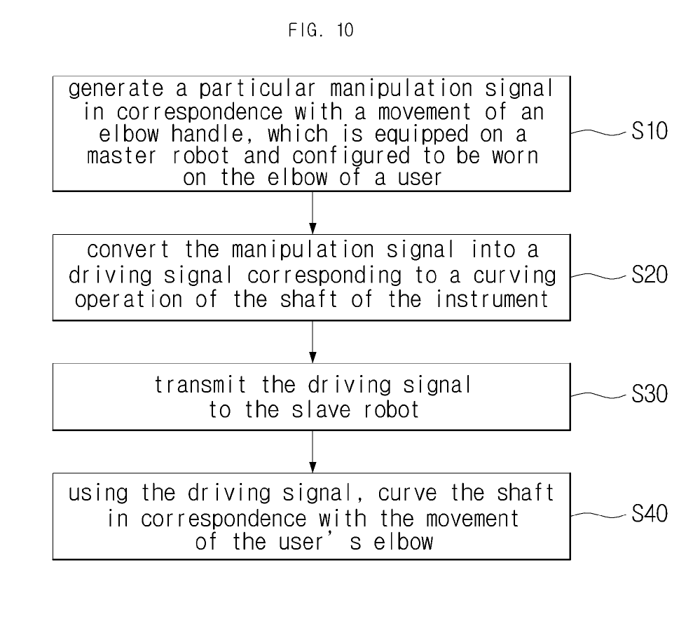

FIG. 10 is a flowchart illustrating a method of driving a surgical robot according to an embodiment of the present invention. This embodiment relates to a method of driving an instrument 10 mounted on a slave robot 7 by manipulating the master interface 3 described above.

That is, this embodiment provides a method of driving an instrument 10, which has a curvable shaft 30, and which is mounted on a slave robot 7, by manipulating a master robot 1 connected to the slave robot 7. First, the separate elbow handle 5 installed on the master interface 3 may be manipulated. The elbow handle 5 is a dedicated handle included in the master interface 3 that is configured to be worn on the elbow of a user. In correspondence with the movement of the elbow handle 5, a particular manipulation signal may be generated (S10).

The generated manipulation signal may be converted into a particular driving signal that corresponds to a curving operation of the shaft 30 (S20), and the converted driving signal may be transmitted to the slave robot 7 (S30), allowing the shaft 30 of the instrument 10 to operate in correspondence with the manipulation of the elbow handle 5. Thus, in an instrument 10 according to this embodiment, the shaft 30 may undergo a curving movement according to the movement of the elbow of the user manipulating the master interface 3 (S40). In this way, a user may intuitively manipulate the instrument 1 on a surgical robot according to this embodiment, just as if the user were moving his or her own arm.

The driving method for the surgical robot described above can also be implemented in the form of a computer program that is read and executed by a digital processing device, such as a microprocessor, etc., which may be either built into the robot itself or connected to the robot from an external source.

While the present invention has been described with reference to particular embodiments, it will be appreciated by those skilled in the art that various changes and modifications can be made without departing from the spirit and scope of the present invention, as defined by the claims appended below.

What is claimed is:

1. A surgical instrument comprising:
a guide member inserted into a surgical site as a surgical trocar, the guide member having an elbow formed in a middle portion thereof;
a driving wheel joined to the guide member, the guide member configured to curve by a particular angle at the elbow by manipulating the driving wheel;
a driving part having a driver in correspondence with the driving wheel;
a core joined to the driving part and extending along one direction, the core configured to be inserted into a surgical site through the guide member; and
an effector joined to a far end of the core and configured to operate in correspondence with a user manipulation on the driving part,
wherein the driving wheel is operated in linkage with a manipulation of the driver by inserting the core into the guide member and mechanically connecting the driving wheel to the driver,
wherein the core is made from a flexible material, and is configured to curve at the elbow as the guide member is curved.

2. The surgical instrument according to claim 1, wherein the elbow includes a hinge axis and an expandable part, the hinge axis formed on one side of a cross section of the guide member, the expandable part formed on the other side of a cross section of the guide member, and wherein the guide member is configured to curve at the hinge axis in a direction that compresses the expandable part.

3. The surgical instrument according to claim 2, wherein the expandable part comprises an elastic body configured to apply an elastic force in a direction that expands or compresses the expandable part.

4. The surgical instrument according to claim 1, wherein a plurality of elbows are formed in the guide member.

5. The surgical instrument according to claim 4, wherein the plurality of elbows are formed such that the guide member is curved in opposite directions, so that the effector moves closer to the driving part as the guide member is curved.

6. The surgical instrument according to claim 1, wherein the driving wheel is connected to the guide member, and the guide member is curved at the elbow by a tensional force applied on the wire by operating the driving wheel.

7. The surgical instrument according to claim 6, wherein the wire is installed such that the wire is exposed at a surface of the guide member, and the wire is pulled out of the guide member as a tensional force is applied on the wire to curve the guide member.

8. The surgical instrument according to claim 7, wherein the guide member has a cylindrical shape, and the wire forms a portion of a perimeter of the guide member.

9. The surgical instrument according to claim 6, wherein the guide member is formed with a channel processed in a cross section thereof, the channel configured to hold the wire.

10. The surgical instrument according to claim 1, wherein the driving part is coupled to a surgical robot arm to be manipulated by a driving force transferred from the robot arm.

11. The surgical instrument according to claim 1, wherein the driving part is formed as a handle to be manually manipulated by a user.

12. A method of driving a surgical instrument mounted on a slave robot by connecting the slave robot to a master robot and manipulating the master robot, the method comprising:
generating a particular manipulation signal in correspondence with a movement of an elbow handle included on the master robot, the elbow handle configured to be worn on an elbow of a user;
converting the manipulation signal into a driving signal corresponding to a curving operation of a shaft of the instrument; and
transmitting the driving signal to the slave robot.

13. The method according to claim 12, further comprising, after the transmitting:
curving the shaft to correspond with a movement of the elbow of the user, using the driving signal.

14. A recorded medium readable by a surgical robot, tangibly embodying a program of instructions executable by the surgical robot for driving a surgical instrument mounted on a slave robot connected to a master robot according to a manipulation of the master robot, the program comprising:
generating a particular manipulation signal in correspondence with a movement of an elbow handle included on the master robot, the elbow handle configured to be worn on an elbow of a user;
converting the manipulation signal into a driving signal corresponding to a curving operation of a shaft of the instrument; and
transmitting the driving signal to the slave robot.
